(19) United States
(12) Patent Application Publication
(10) Pub. No.: US 2004/0082840 A1
(43) Pub. Date: Apr. 29, 2004

(54) Health monitor expansion module and sensor module

(75) Inventor: Tse Min Chen, Taipei (TW)

Correspondence Address: BACON & THOMAS, PLLC, 625 SLATERS LANE, FOURTH FLOOR, ALEXANDRIA, VA, 22314

(73) Assignee: Institute for Information Industry, Taipei (TW)

(21) Appl. No.: 10/601,595

(22) Filed: Jun. 24, 2003

(30) Foreign Application Priority Data
Oct. 24, 2002 (TW) 91124728

(51) Int. Cl. A61B 5/00

(52) U.S. Cl. 600/300; 128/903; 340/539.12

(57) ABSTRACT

A health monitor expansion module may be combined with a mobile apparatus to provide a wireless health monitoring platform. The health monitor expansion module stores a patient's personal health data for easy carrying. Furthermore, the present invention provides a sensor module which can be connected to a physiological sensor to send a physiological signal to the health monitor expansion module.

### BACKGROUND OF THE INVENTION

1. Field of the Invention

The present invention discloses hardware and a related software program for monitoring a patient's health condition.

2. Description of the Related Art

Because a patient's health condition may suddenly change, the prior art physiological monitor is capable of providing (through a GPS system) the patient's position to a hospital, a doctor or an emergency room. Medical workers can then send their staff to the patient's position.

However, even though the patient is utilizing physiological sensors (such as a heart sensor, a blood pressure sensor, or a thermometer), all of these sensing signals are only provided for reference, and are not automatically sent to the hospital or emergency room. When the patient undergoes immediately physiological changes, the hospital or emergency room may not be properly prepared.

Furthermore, the prior art sensors have a wired connection with the portable host, which limits their usage and convenience.

In addition, when using the prior art sensors, each patient's history of physiological data may be stored in different computers, and so the portable apparatus may not be convenient in this regard.

Therefore, it is desirable to provide an improved health monitor expansion module and sensor module to mitigate and/or obviate the aforementioned problems.

### SUMMARY OF THE INVENTION

A main objective of the present invention is to provide a health monitor expansion module which can be combined with a mobile apparatus to provide a wireless health monitoring platform.

Another objective of the present invention is to provide portable health history data, which can be easily provided to a doctor.

Another objective of the present invention is to provide a sensor module which can be connected to a physiological sensor to send a physiological signal to the health monitor expansion module via a wireless connection.

In order to achieve the above-mention objectives, the health monitor expansion module of the present invention includes a microprocessor, an input signal processing unit connected to the microprocessor for receiving and processing the signal provided by the sensor module; and an interface processing unit for processing transmitted signals between the mobile apparatus.

The sensor module of the present invention includes a microprocessor, an input signal processing unit connected to the microprocessor for receiving and processing the signal provided by the sensor, and an antenna and a wireless data transmitting unit for sending the physiological signal to the health monitor expansion module.

Other objectives, advantages, and novel features of the invention will become more apparent from the following detailed description when taken in conjunction with the accompanying drawings.

### BRIEF DESCRIPTION OF THE DRAWINGS

FIG. 1 is an environment schematic drawing of the present invention.

FIG. 2 is a function block drawing of a sensor module of the present invention.

FIG. 3 is a function block drawing of a health monitor expansion module of the present invention.

FIG. 4 is a flowchart of a portable apparatus of the present invention.

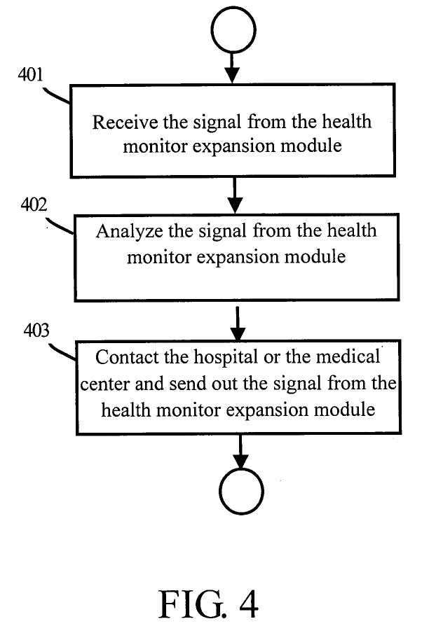

FIG. 5 is a flowchart of the health monitor expansion module of the present invention.

### DETAILED DESCRIPTION OF THE PREFERRED EMBODIMENT

Illustrative embodiments of the invention are described below. In the interest of clarity, not all features of an actual implementation are described in this specification.

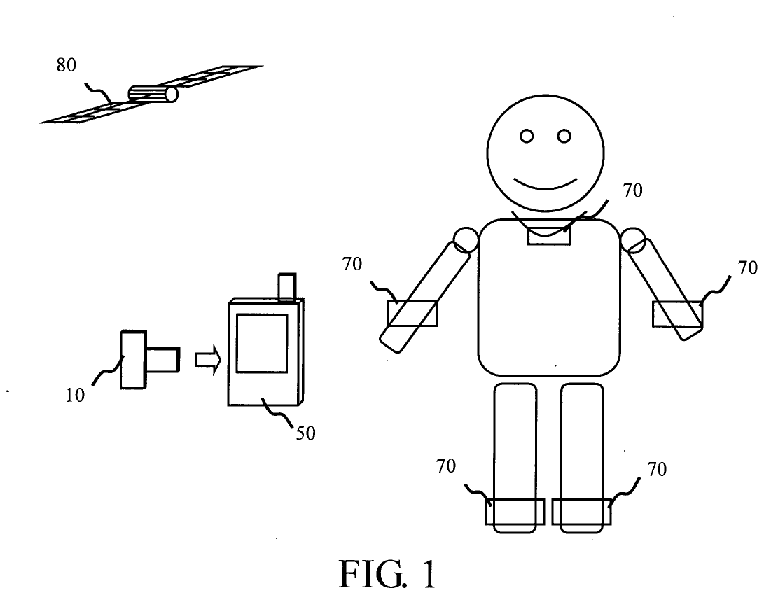

Please refer to FIG. 1. FIG. 1 is an environment schematic drawing of the present invention. A patient 90 utilizes a plurality of sensor modules 70. The sensor modules 70 can send related physiological signals of the patient 90 to a health monitor expansion module 10. The health monitor expansion module 10 can be combined with a portable apparatus 50 to serve as a wireless health monitoring platform 20; the health monitor expansion module 10 receives the signals from the sensor modules 70. The portable apparatus 50 has wireless communications ability, such as a mobile telephone, a portable computer with wireless communications functionality (for example, a PDA), etc. If a sensor module 70, or the portable apparatus 50, has a GPS module, then the patient's position can be determined from a signal sent from a satellite 95.

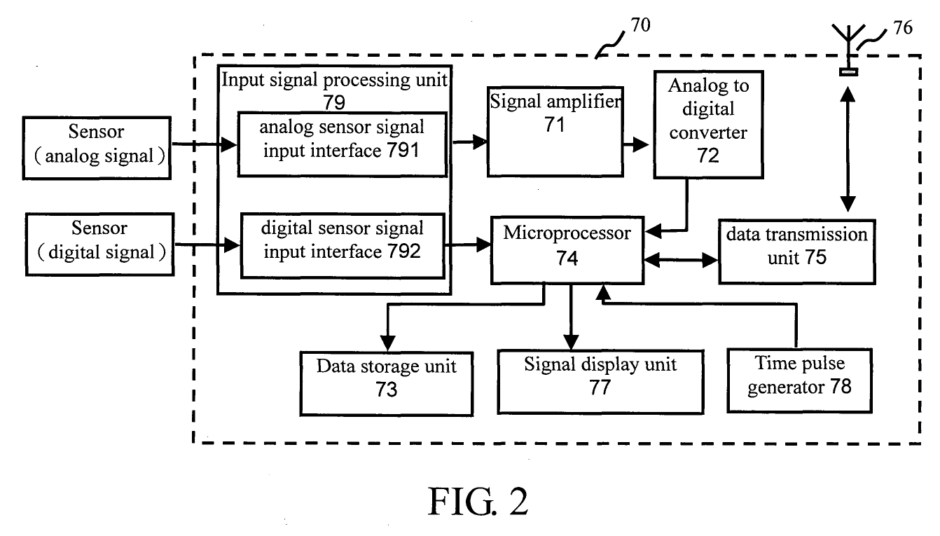

FIG. 2 is a function block drawing of a sensor module 70 of the present invention. The sensor module 70 includes a signal amplifier 71, a digital to analog converter 72, a data storage unit 73, a microprocessor 74, a wireless data transmitting unit 75, an antenna 76, a signal display unit 77, a time pulse generator 78 and an input signal processing unit 79. The input signal processing unit 79 has an analog sensor signal input interface 791 and a digital sensor signal input interface 792. The sensor module 70 can be internally supplied with power, for example by way of a battery (not shown), or externally supplied with power by an external apparatus.

If the sensor 80 outputs an analog signal, this analog signal may be sent into the analog sensor signal input interface 791, then to the signal amplifier 71 to amplify the signal, and finally the signal can be converted to a digital signal by way of the digital to analog converter 72 to be received by the microprocessor 74.

If the sensor 80 outputs a digital signal, the digital signal may be sent to the microprocessor via the digital sensor signal input interface 792.

The data storage unit 73 is connected to the microprocessor 74, and is used for storing data, such as a software program or data output by the sensor 80. The data storage unit 73 can be a flash memory or other non-volatile memory.

The signal display unit 77 can be an LED (not shown) to indicate if the sensor module 70 is in a normal operating status.

As the time pulse generator 78 is a basic digital circuit element, no more explanation is needed here.

The wireless data transmission unit 75 and the antenna 76 can send the signal from the sensor module 70 to the health monitor expansion module 10.

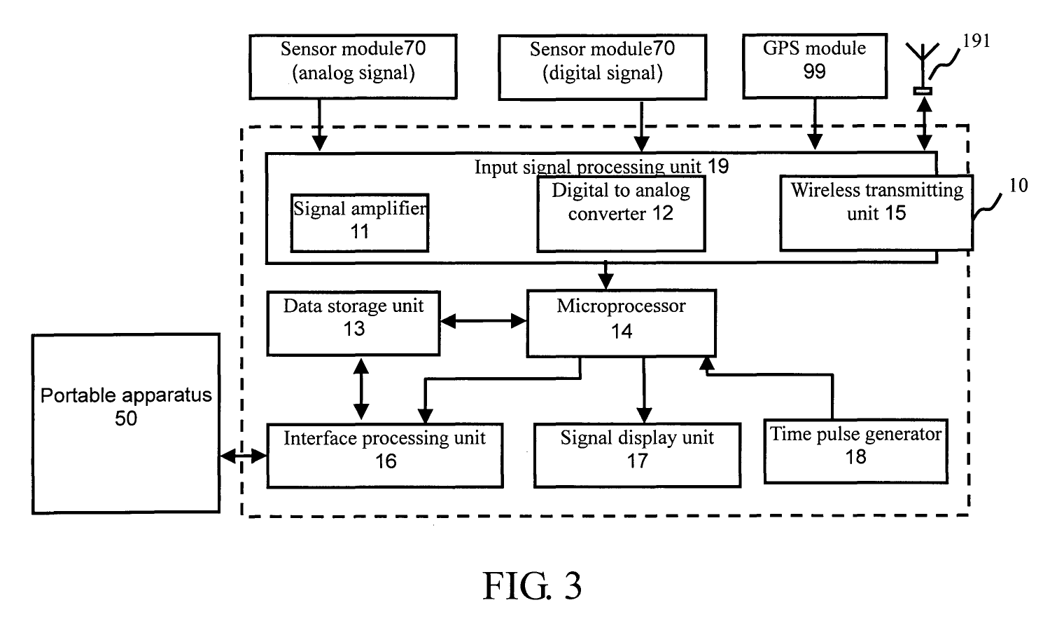

FIG. 3 is a function block drawing of a health monitor expansion module 10 of the present invention. The health monitor expansion module 10 comprises a data storage unit 13, a microprocessor 14, an interface processing unit 16, a signal display unit 17, a time pulse generator 18, an input signal processing unit 19 and an antenna 191. The input signal processing unit 19 has a signal amplifier 11, a digital to analog converter 12 and a wireless transmitting unit 15.

The input signal processing unit 19 can receive the signal from the sensor module 70 via a wired connection. If the sensor module 70 transmits the signal in a wireless manner, the signal may be sent through the antenna 191 and the wireless transmitting unit 15. Furthermore, the health monitor expansion module 10 can not only receive the signal from the sensor module 70, but also the signal from the GPS module 99, and so the portable apparatus 50 can determine the position of the patient and send this position.

If the sensor module 70 outputs an analog signal, the signal may be sent into the signal amplifier 11 to be amplified and converted into a digital signal via the digital to analog converter 12 to be received by the microprocessor 14. If the sensor module 70 outputs a digital signal, the signal may be sent to the input signal processing unit 19 to be passed on to the microprocessor 14

The data storage unit 13 and the microprocessor 14 are connected to each other, and provide for the storing of data, such as a software program or data output by the sensor module 70. The data storage unit 13 can be a flash memory or other non-volatile memory.

The signal display unit 17 can be an LED (not shown) to indicate if the health monitor expansion module 10 is in a normal operating status.

The time pulse generator 18 is a basic digital circuit element, and so no more explanation is required here.

The interface processing unit 16 is used for processing the transmitted signals between the portable apparatus 50. The interface processing unit 16 can be a PCMCIA, SDIO or CF interface, all of which are general standards suitable for the portable apparatus 50.

The hardware structure of the portable apparatus 50 is identical with that of a small computer (in particular, like that of a mobile telephone), but such structure is not the key point of this present invention, and so there will be no more explanation of it here. The primary function of the portable apparatus is to receive the signal from the health monitor expansion module 10 (step 401), after the installation of an appropriate software program. The software program of the portable apparatus 50 may additionally perform other more complicated functions; for example, the software may analyze the signal from the health monitor expansion module 10 (step 402), and if the signal shows a dangerous condition, the portable apparatus 50 will contact the hospital or the medical center and send out the signal from the health monitor expansion module 10 or the detected dangerous signal (step 403).

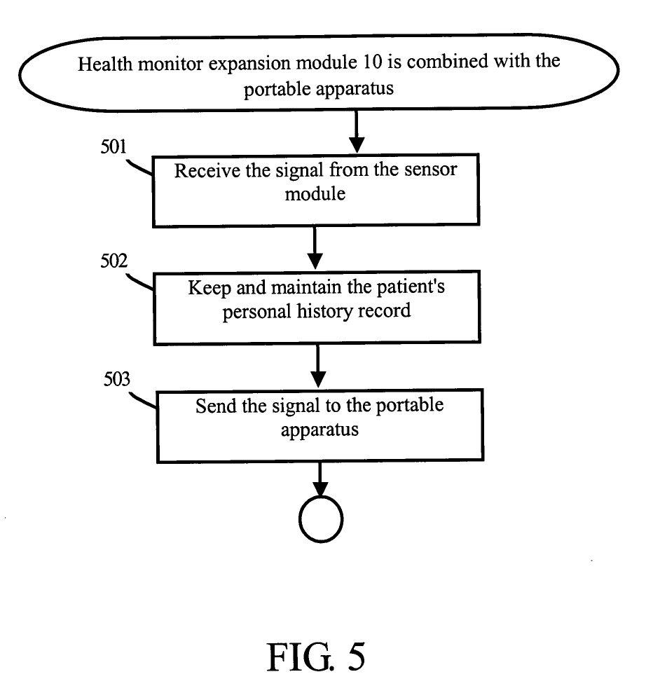

Please refer to FIG. 5. After the health monitor expansion module 10 is combined with the portable apparatus 50, the portable apparatus 50 can supply power to the health monitor expansion module 10, and the health monitor expansion module 10 can initialize its software and hardware. The health monitor expansion module 10 can then command the sensor module 70 to transmit the signal, or the sensor module 70 can automatically send the signal to the health monitor expansion module 10 with every predetermined time interval (step 501). The health monitor expansion module 10 stores the signal from the sensor module 70 to keep and maintain the patient's personal history record (step 502). The health monitor expansion module 10 can send the signal to the portable apparatus 50 according to a request (especially when the portable apparatus 50 detects a dangerous physiological condition), or send the signal to the portable apparatus 50 with every predetermined time interval (step 503).

The invention has been described using exemplary preferred embodiments. However, for those skilled in this field the preferred embodiments can be easily adapted and modified to suit additional applications without departing from the spirit and scope of this invention. Thus, it is to be understood that the scope of the invention is not limited to the disclosed embodiments. On the contrary, it is intended to cover various modifications and similar arrangements based upon the same operating principle. The scope of the claims, therefore, should be accorded the broadest interpretations so as to encompass all such modifications and similar arrangements.

Although the present invention has been explained in relation to its preferred embodiment, it is to be understood that many other possible modifications and variations can be made without departing from the spirit and scope of the invention as hereinafter claimed.

What is claimed is:

1. A health monitor expansion module for connecting to a mobile wireless communication apparatus to provide a wireless health monitor platform, the health monitor expansion module capable of receiving physiological signal provided by at least one sensor module, the health monitor expansion module comprising:
a microprocessor;
an input signal processing unit connected to the microprocessor for receiving and processing the signal provided by the sensor module;
a data storage unit connected to the microprocessor for storing data; and
an interface processing unit for processing transmission signals between the mobile apparatus;
wherein the health monitor expansion module is capable of receiving the physiological signal from the sensor module and send the physiological signal via the mobile apparatus.

2. The health monitor expansion module as claimed in claim 1 further comprising an antenna and a wireless data transmitting unit to enable the health monitor expansion module to receive the wireless output signal from the sensor module.

3. The health monitor expansion module as claimed in claim 1 further comprising a digital-to-analog converter to enable the health monitor expansion module to receive an analog output signal from the sensor module.

4. The health monitor expansion module as claimed in claim 1 further comprising a signal amplifier for amplifying the signal from the sensor module.

5. A sensor module capable of connecting to a physiological sensor to send the physiological signal to the health monitor expansion module as claimed in claim 1, the sensor module comprising:
a microprocessor;
an input signal processing unit connected to the microprocessor for receiving and processing the signal provided by the sensor;
a data storage unit connected to the microprocessor for storing data; and
an antenna and a wireless data transmitting unit for sending the physiological signal to the health monitor expansion module.

6. The sensor module as claimed in claim 5 further comprising a digital-to-analog converter to enable the health monitor expansion module to receive the analog output signal from the sensor.

7. The sensor module as claimed in claim 5 further comprising a signal amplifier for amplifying the signal from the sensor.
